(19) United States
(12) Patent Application Publication
(10) Pub. No.: US 2025/0072818 A1
(43) Pub. Date: Mar. 6, 2025

(54) Method for Measuring Periodic Headpulse Sleep Bursts to Determine Brain Health

(71) Applicant: The Regents of the University of California, Oakland, CA (US)

(72) Inventors: Paul A. Lovoi, Los Altos, CA (US); Wade Smith, Larkspur, CA (US)

(73) Assignee: The Regents of the University of California, Oakland, CA (US)

(21) Appl. No.: 18/821,908

(22) Filed: Aug. 30, 2024

Related U.S. Application Data
(60) Provisional application No. 63/580,325, filed on Sep. 1, 2023.

(51) Int. Cl. A61B 5/00; A61B 5/024; A61B 5/0245; A61B 5/16; G16H 50/70

(52) U.S. Cl. CPC A61B 5/4064; A61B 5/165; A61B 5/4088; A61B 5/4812; A61B 5/6803; A61B 5/7264; G16H 50/70; A61B 5/02416; A61B 5/0245; A61B 2560/045; A61B 2562/0219

(57) ABSTRACT

In a normal human being the pulsing of blood through the brain during sleep causes a periodic headpulse of high amplitude, detectable and measurable using cranial accelerometry. A patient's sleeping headpulse is compared to one or more reference headpulse patterns from normal and abnormal patients to determine health and condition of the brain and vasculature.

This application claims benefit of provisional application Ser. No. 63/580,325, filed Sep. 1, 2023.

### BACKGROUND AND SUMMARY OF THE INVENTION

This invention concerns cranial vasculature health and assessment of health or disorder, and in particular concerns use of a recently discovered signal caused by pulsing of blood through the head during sleep.

What is called herein the headpulse is a novel biological phenomenon measurable in mammals, notably humans, involving a mechanical force on the head and brain produced by the cardiac contraction and subsequent blood flow into the brain. The headpulse has been measured by a device attached against the head, specifically a highly sensitive accelerometer, providing a time-domain varying signal. The same device has been used to measure headpulse of LVO (large vessel occlusion) stroke patients and has been useful in determining conditions of stroke and possible indications that a stroke could occur. Even in dogs the headpulse has been detected.

The invention is concerned with measuring the headpulse during sleep, using cranial accelerometry.

Just prior to sleep, after a varying latency from sleep onset (e.g. 3 to 20 minutes) the accelerometer device reveals intermittent dyschronous forces or “bursts” with 1 to 5 spikes that are higher in amplitude than the surrounding headpulse background by as much as 3 to 5 times the amplitude of the typical interburst levels. The spikes can be are spaced 3-5 seconds apart with the entire burst up to about 20 seconds. In many cases these sleep bursts occur regularly at a period fixed for the individual subject, but typically every 20-50 seconds while they are occurring (there may be periods without the bursts). These recurrent bursts in headpulse power can be called Headpulse Sleep Bursts (HPSB).

The burst begins just after sleep onset, and typically maintains constant throughout the night, ceasing somewhat before the subject awakens because of some stimulus, such as needing to visit the bathroom, or an alarm or other sound that has not yet awakened the subject.

This phenomenon is believed to represent glymphatic functions of the mammalian brain, indicating that the brain uses the force resulting from the higher headpulse spike to physically pressurize the brain to expel interstitial fluids into the perivenular spaces. It is a significant force being at least three times normal amplitude, thus involving significant pressure in the brain. There are no known non-invasive methods to measure human brain glymphatic functions. This is the only known technique to non-invasively measure human brain glymphatic function.

Brain glymphatic function may be the central reason why mammals sleep, and dysfunction of this system has been hypothesized to be a causal factor in human neurodegenerative disease. Measurements of patients with mild cognitive impairment (MCI) has shown a dramatic decrease in the number of sleep spikes during sleep. Measuring sleep bursts, and quantifying their frequency, number, power and individual variances, then provides non-invasive information that predicts the onset or progression of neurodegenerative disorders, and therefore may be used to diagnose sleep function and disfunction. Use of the device will have important application as a research device for use by sleep researchers.

The sleep bursts are a novel biological marker that appears only during sleep, and it may have several yet unexpected and unknown uses in health and disease. The signal has been detected and measured in every subject tested. Every one of dozens of tested subjects with no neurological disease exhibits this phenomenon. Variance from the typical burst pattern of a healthy person may be an important diagnostic tool.

Sleep bursts or spikes, their presence or absence, and characteristics, may be used as a biological marker, especially when detected by a wearable device with a three-axis accelerometer, as follows:
a. A quantitative method to characterize the quality of human sleep (by calculating the number and frequency of sleep spikes during a normal night of sleep)
b. To determine if sleep is disruptive so that sleep spikes or bursts don't occur, thereby indicating that brain cleaning and healing has not occurred
c. A method of determining when not to wake up a hospitalized patient so as to not disturb the period of sleep that includes sleep spikes or bursts
d. Using the sleep burst power to aid in the diagnosis of sleep disorders including insomnia, sleep apnea and parasomnias
e. As a predictor of the risk of developing neurodegenerative disorders inclusive of Alzheimer's Disease, Parkinson's Disease, Frontotemporal Dementia, and Picks Disease and any other form of dementia that share the common pathology of storage proteins (a-beta, alpha synuclein, tau, etc.)
f. As a method of treating these disorders by pharmacotherapy (drugs) that restore sleep spikes or bursts. Sleep spikes or bursts can be markers of drug effectiveness for these diseases. Current methods of judging whether or not a drug is working for neurodegenerative disease take years to see an effect. Sleep spikes and bursts provide a nearly immediate response that cuts drug development time
g. As a method to measure the influence of stress on sleep dysfunction
h. As a method to follow response to cognitive behavioral therapy and pharmacotherapy for sleep induction and sleep maintenance

### DESCRIPTION OF THE DRAWINGS

FIG. 1 is a schematic elevation view indicating a subject wearing a headset sensor and indicating the system of the invention.

FIG. 2 is a block diagram indicating the system.

FIG. 3 is a perspective view showing a sleeping subject with the head-worn device.

FIGS. 4, 5, 6, 7 and 8 are graphs with waveforms indicating a HeadPulse signal that typically includes a periodic spike signal observed during sleep for different subjects, in accordance with the invention.

### DESCRIPTION OF PREFERRED EMBODIMENTS

The accelerometry data derived according to the invention, energy versus time for multiple heartbeats, is referred to herein as the HeadPulse. The heartbeat is the period of one cardiac cycle, corresponding to the R-R interval (ECG QRS peak representing the peak of systole) from one heartbeat to the next. The heartbeat, i.e. the R-R interval, can be used to parse the HeadPulse recording into multiple data samples wherein each data sample represents the accelerometer in a time domain for one heartbeat.

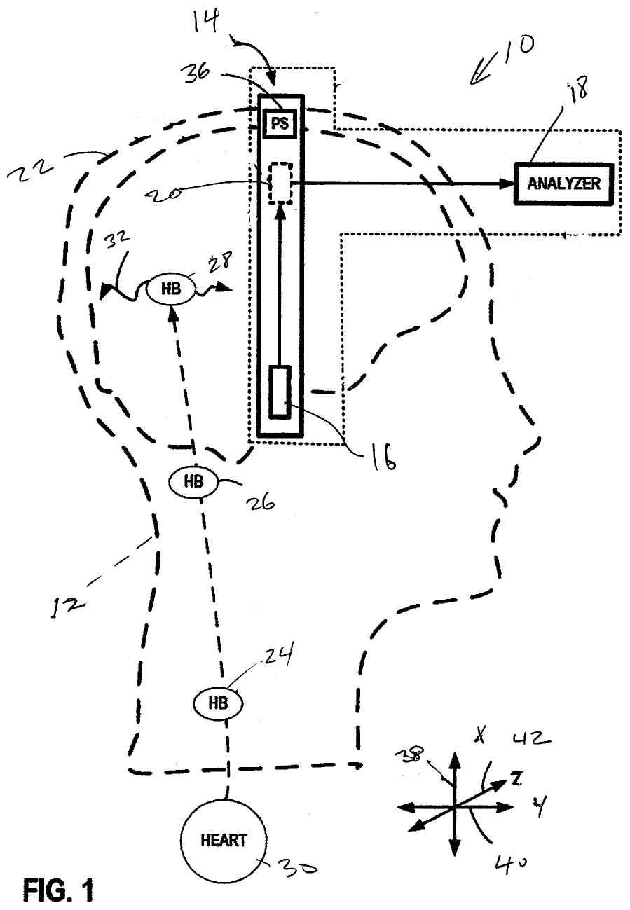

In the drawings, FIG. 1 illustrates one example of a system 10 for observing the HeadPulse of a subject 12, i.e. observing head acceleration due to heartbeat, to record and evaluate brain conditions. In the examples discussed herein, the system 10 detects and records a headpulse sleep burst signal that occurs in a patient or subject during sleep. As discussed above, this burst occurring typically at intervals of about 20 or 30 to about 50 seconds. The burst signal, which does occur after a series of heartbeats, appears to relate to an important housekeeping function of the brain, possibly wherein the glymphatic function of the brain removes cells or interstitial fluids that are to be “cleaned out” during sleep.

In FIG. 1 the system 10 includes a headset 14 worn against the head of the subject. This can be a headband or any device that is affixed to the head sufficiently securely that the device will remain in place during a night of sleep. On the headset is an accelerometer 16, engaged against the cranium. Some form of securing mechanism retains the headset device 14 securely against the head such that the accelerometer 16 is substantially fixed in place.

As indicated in the diagram, the system includes an analyzer 18, which can be onboard the headset 14 or separate, communicating to receive data from the headset. A communication interface 20 is indicated on the headset, for an embodiment in which the analyzer 18 is separate from the headset and wirelessly connected. In another embodiment data can be stored in memory in the headset 14, and communicated later, such as after the patient awakens, to the analyzer 18. In a specific embodiment the analyzer 18 is contained in a smartphone and receives data wirelessly from the headset. In such an embodiment the transfer of data to the analyzer 18 need not be wireless, but can be by a wired connection to the headset after the period of sleep analysis. Alternatively, the data can be stored on a removable memory, such as a memory card, flash card, memory stick, etc., and can be moved to the analyzer 18 to transfer the data at an appropriate time.

Other arrangements are also possible, and it should be understood that the headset can be self-contained, so as to be connectable to a smartphone either by wire or wirelessly after a sleep session, or data can be sent contemporaneously to the smartphone throughout the sleep session. Another arrangement is for the analyzer 18 to be completely contained on the headset or wearable device in contact with the head.

As is known, the heartbeat produces a force on the head and neck, through the cerebral vasculature. The force on the brain produced by the heartbeat is transferred to the skull. When the subject is prone during sleep, the force of blood entering the brain in the vertical or X-axis direction (vertical referring to the head when upright), can more freely move the brain and other tissues, not being restrained by gravity along that axis. The head 22 of the subject 12 moves in response to each heartbeat, heartbeats being indicated at 24, 26 and 28 and the heart being indicated at 30. In the illustrated system the accelerations (indicated at 32) of the head are detected with the accelerometer 16, ultimately to enable a plot with a waveform of accelerations/head movements against time with the acquired data.

A power supply for the headset is indicated at 36.

The accelerometer 16 detects acceleration in at least one direction or axis, and generates an accelerometer signal based on the acceleration of the head. The most important axis for purposes of this invention is the vertical axis relative to a standing person, denoted herein as the X axis 38 in FIG. 1. Other axes are the Y axis 40 and the Z axis 42, for movement in the forward-back direction and in the left/right direction, respectively.

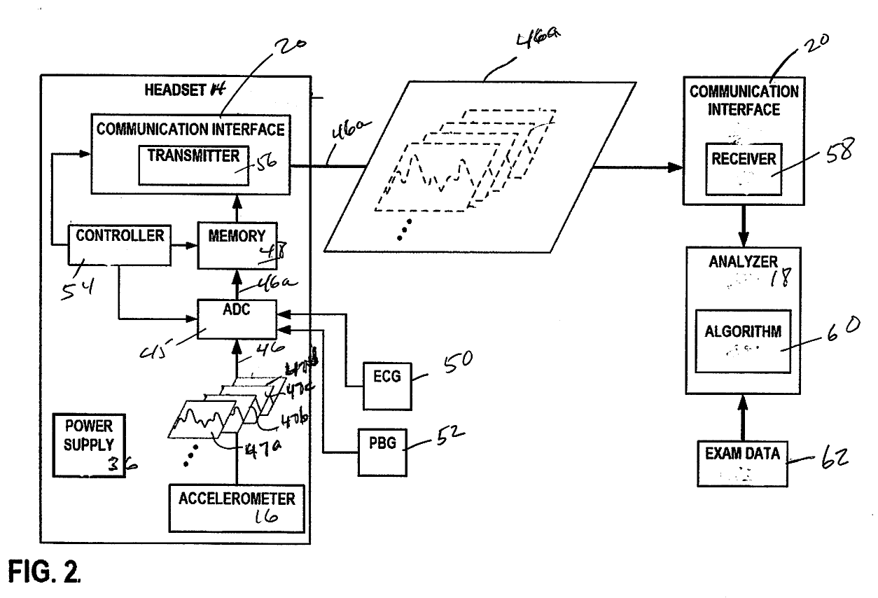

The headset is represented in the block diagram of FIG. 2. Data representing the acceleration of the head can be conveyed in several ways. In FIG. 2 it is shown that the analog data output 46 of the accelerometer 16, with individual heartbeat data indicated as 47a-47d, is converted by an ADC 45 to digital data 46a that is periodically transmitted to the analyzer 18, indicative of acceleration over time for a number of heartbeats. The digital data are stored in a memory unit 48. In one embodiment, signals from an electrocardiography device (ECG) 50 can be input to the ADC 45 along with the data. A PPG signal 52 could be used, i.e. a photoplethysmograph, one of these signals used to set a heartbeat time reference for the acceleration signals. PPG is a waveform that resembles the arterial pulse. In another embodiment the ECG and/or PPG signal can be omitted, since the timing of the heartbeats can be derived from the accelerometry waveforms or data samples obtained by opposite head positions of accelerometers. Other suitable known techniques may be used to obtain heartbeat timing data. Analysis of the waveform can be accomplished without the use of heartbeat timing.

A controller 54 is also shown in FIG. 2, which can be any suitable type of processor, logic circuit or other arrangement that manages the functions described herein, facilitating the overall functionality of the headset. The controller is shown as providing instructions to the memory 48 and the ADC 45 in the drawing. The controller 54 is also shown as controlling the communication interface 20, which includes a transmitter 56.

Converted digital data 46a are retrieved from the memory 48 and transmitted to the analyzer 18, which can be separate from the headset as indicated in the example of FIG. 2. This is via the receiving end of the communication interface on the right side of FIG. 2, also shown as 20 and including a receiver 58. The example indicates a wireless connection for transmission of the data, although a wired connection could be used as described above. Bluetooth or other near-field communication protocol can be used. The analyzer 18 is shown as embodying an algorithm 60. The algorithm can be used, for example, to compare the subject's accelerometer data to accelerometer data from other subjects, particularly subjects known to be healthy and with normal sleep burst waveforms. Such “normal” data is shown as input at the block 62 in FIG. 2.

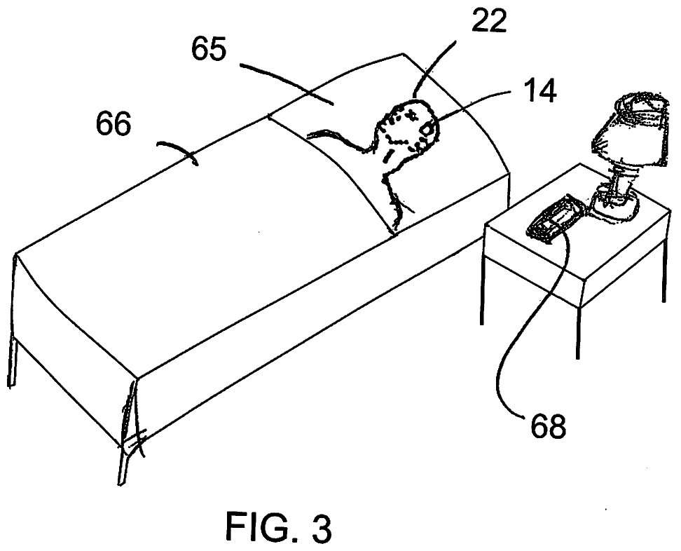

FIG. 3 shows a sleeping subject 65, lying supine in a bed 66. The headset 14 of the invention is shown attached to the patient's head 22. Adjacent to the bed is indicated a smartphone 68, to receive signals wirelessly from the headset 14 representing movements of the head due to heartbeat during sleep. Communication can be by a near field communication protocol such as Bluetooth, for example. The smartphone 68 receives the digital data as indicated in FIG. 2, and the receiver, analyzer and algorithm can be contained in the smartphone 68 in this example. Alternatively, the headset can be a more comprehensive construct, with data analysis performed onboard the headset, and a device such as a computer or smartphone could simply receive the results of the data analysis for review or transmission elsewhere using Wi-Fi or cell signal. Further, the integrated device, with the analyzer or processor on the device, could provide the results directly on a display without the need for a transfer of the data to a smart device.

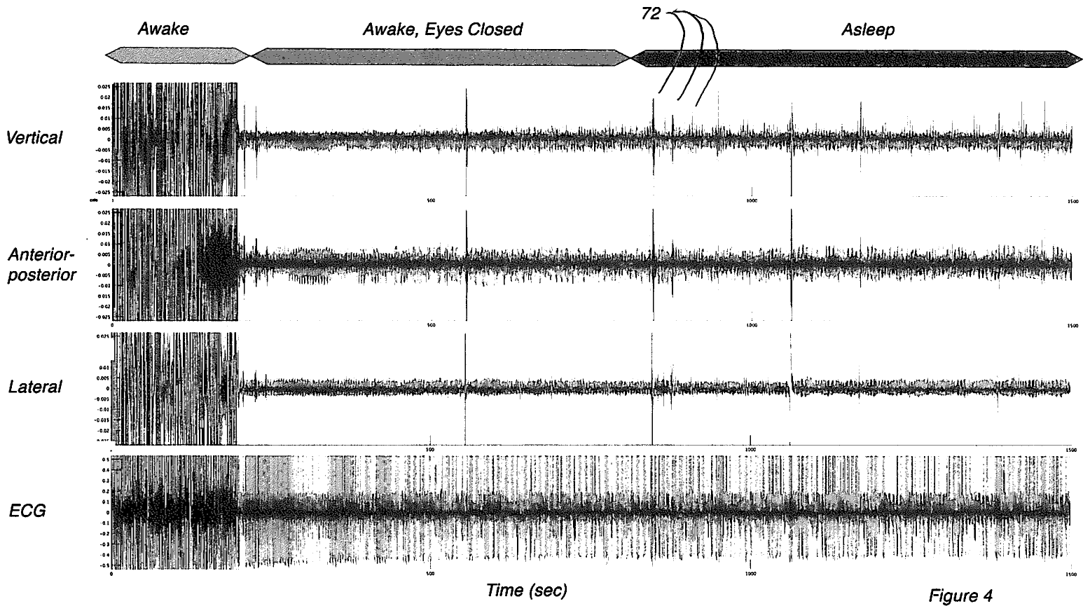

FIG. 4 shows waveforms for accelerometer data in the X (vertical), Y (anterior-posterior) and Z (lateral) axes in a normal subject. As noted in FIG. 1, the X axis is the normally-vertical axis of movement of the head, while the Y axis is the forward/back axis and the Z axis is the left/right axis of movement. At the bottom of the chart of FIG. 4 is a waveform simply showing the heartbeat (electrocardiogram). The subject is first awake and preparing for bed with the headset on; during this activity accelerometer data are erratic and show high magnitude of movement. Then the subject lies awake with eyes closed, then allows himself to fall asleep.

The horizontal axis in the charts is in seconds of time, spanning about three hours, while the vertical axis basically indicates energy of the acceleration movements, in each of the X, Y and Z waveforms. The X waveform, i.e. the vertical acceleration movements of the cranium (vertical referring to a standing person), is the waveform of greatest interest. As noted above, the supine subject has brain and cranial movements, induced by pulse, that are not restrained by gravity in the X axis. Thus, the periodic spike is exhibited most prominently on the X axis. In this example the periodic spikes are noted at 72, that first appear after falling asleep, occurring for this subject approximately at 45 to 50 second intervals (13 or 14 spikes per ten minutes). The subject's heartbeat, displayed in the bottom waveform, is shown in coordination with the X, Y and Z waveforms for timing. The main spikes 72 generally occur every 20 to 50 seconds.

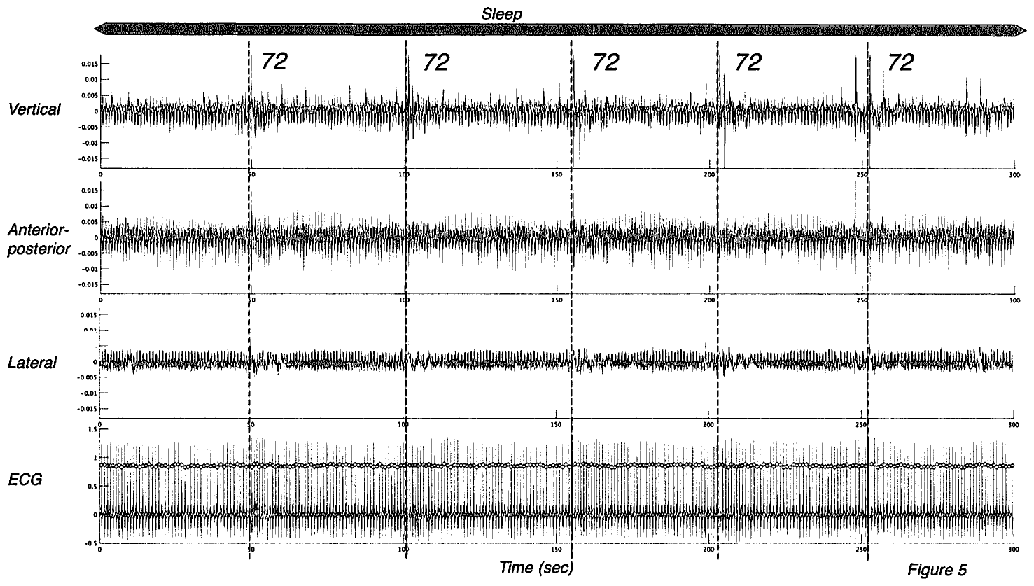

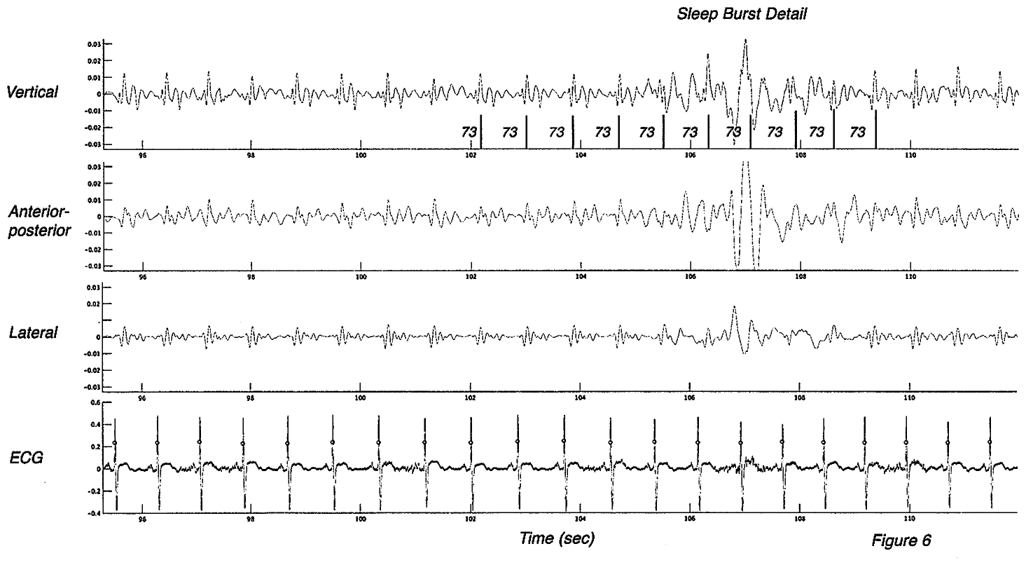

FIGS. 5 and 6 show expanded waveforms, and represent time spans of about 300 seconds and 18 seconds, respectively. FIG. 5 indicates the main spikes 72 with vertical lines since the spikes or bursts occur as small clusters. Other subjects do not have clusters of spikes but simple complex repetitive spikes as shown in FIG. 6. The figure shows the timing of cardiac contractions illustrated by vertical marks 73 revealing a complex superposition of a larger signal in combination with the underlying normal signal. Once the superimposed signal passes, the underlying signal reappears in register with the cardiac contraction. FIG. 6 also shows that the Y and Z axes of movement exhibit less prominent data, with the Z axis providing the least notable data regarding the sleep spike.

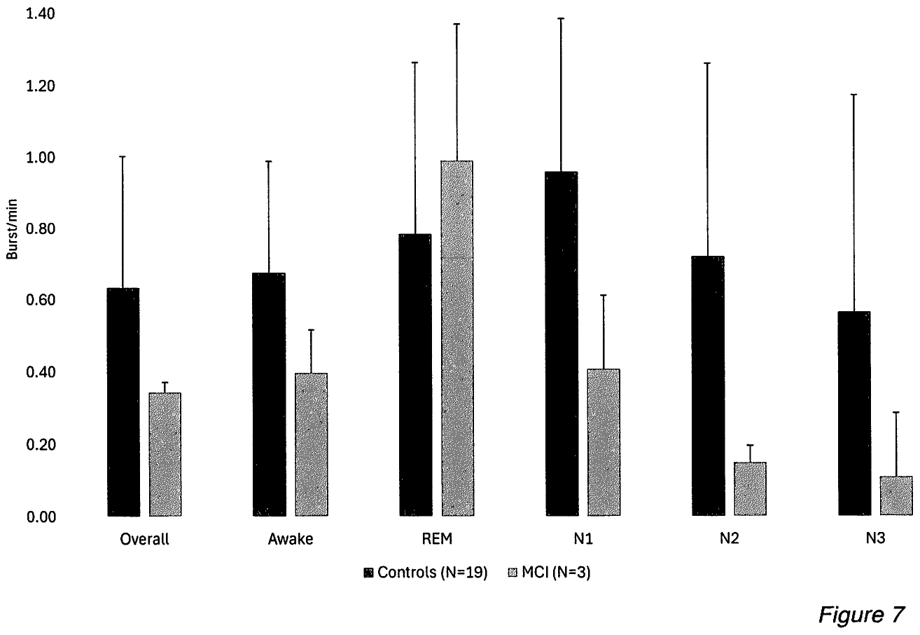

FIG. 7 shows the frequency of sleep bursts during a period of sleep from several normal controls (N=18, 42 recordings) and two patients with mild cognitive impairment (MCI, 3 nights of sleep). The frequency of sleep bursts is defined as the number of sleep bursts during the sleeping period divided by the duration of sleep in minutes. As shown in FIG. 7, overall normal subjects have a burst frequency of 0.62 bursts/min (slower than individual runs of repetitive sleep bursts because this statistic uses total time asleep and there are periods where no sleep bursts appear). MCI patients on the other hand have a significantly lower frequency of 0.36 per minute (P<0.00001). FIG. 7 also shows that sleep bursts are occurring in every stage of sleep, including the awake stage just before sleep and that in normal subjects these occur at a similar frequency. Therefore, sleep bursts in normal subjects occur at every sleep stage which identifies this as a newly discovered physiological parameter of sleep in humans. In addition, FIG. 7 shows that the 3 MCI subject records showed a predominance of sleep bursts during rapid eye movement (REM) sleep (N1, N2, N3). This observation shows that MCI (a precursor of Alzheimer's disease typically) is identifiable by a lower frequency of sleep bursts.

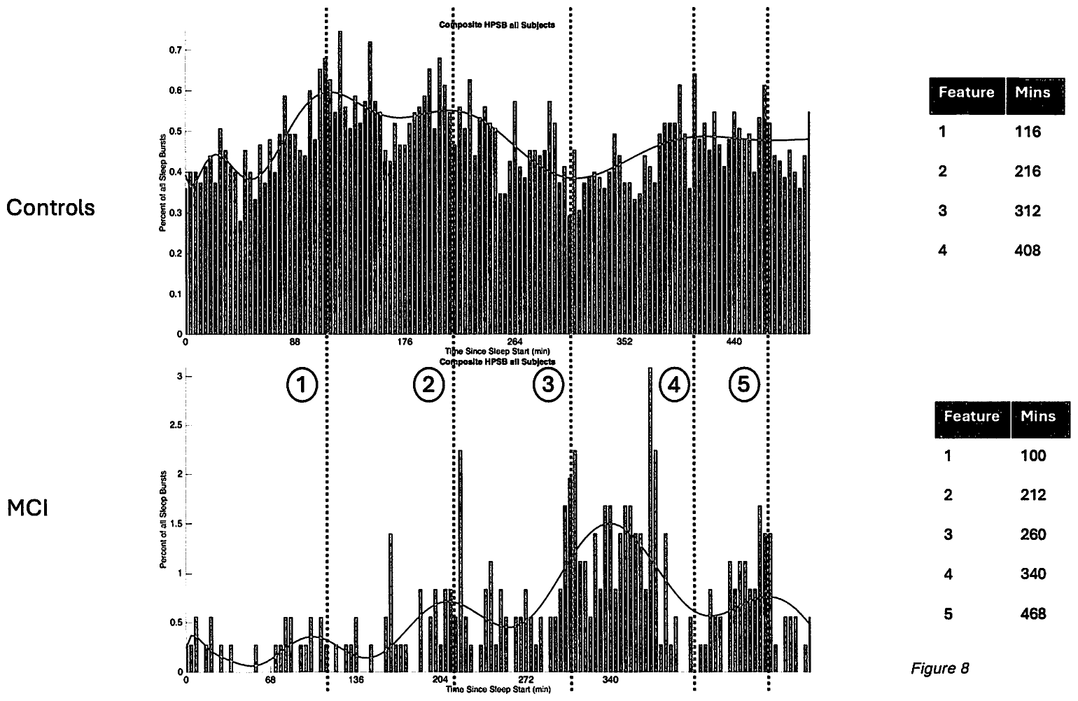

FIG. 8 shows a comparison of when sleep bursts appear in normal subjects compared to those with MCI. In the top panel the group of 18 normal subjects who had 300 minute or more sleep recordings are represented in a histogram from time of sleep onset where sleep bursts occurred. This shows a predominance at approximately 100 and 200 minutes of sleep, a suppression at 300 minutes and another predominance at 400 minutes. MCI patients shown in the lower histogram have a different profile with a clustering of sleep bursts mostly at 300-400 seconds—the time that normal subjects are suppressed—and another increase some 75 minutes later. This pattern of temporal occurrence of sleep bursts following sleep onset is a principal distinction between normal subjects and subjects with mild cognitive impairment. This observation allows the medical device in FIG. 1 to be used as a diagnostic and prognostic tool for separating normal subjects with cognitive complaints from other cause (anxiety, medications, etc.) from those with neurodegenerative disorders.

As explained above, the sleep spike or burst phenomenon appears connected with housekeeping functions of the brain that normally occur during sleep, such as cleaning out of interstitial fluid and cells and to refresh the brain during sleep. It is believed that a patient with a brain disorder, which may be dementia, concussion, moderate to severe traumatic brain injury, or other serious neurodegenerative disorders or conditions, or even insomnia, will exhibit either a lack of sleep burst, maldistribution of sleep bursts or significantly greater intervals between sleep bursts.

It is theorized that the sleep bursts or spikes occur only at intervals of 20 to 50 seconds to provide a brain cleansing function. By reporting the number of sleep bursts one can report the quality of brain cleaning during a night of sleep.

The invention can enable analysis of a subject during sleep, with an investigation of sleep spikes to determine whether the subject may be suffering from a disease or abnormal brain condition or function. It can document the consequences of sleep description and the quality of sleep recovering on subsequent sleep episodes. It can inform a person if a nap was beneficial. It may be used as a method to tell if someone is having microsleeps (brief episodes of sleep during the day), as would be useful to know prior to driving a car or flying a plane. It can be useful to tell whether a person's complaint of memory loss is due to a neurodegenerative disease or psychiatric disease or medication side effect.

### Report of Testing

Materials and Methods: Adult volunteers consented to have headpulse and sleep profiling measurements during normal sleep. The headpulse was recorded from a custom UCSF-designed device worn as a hairband containing force transducers in contact with the temporal scalp anterior to the right ear. These signals were digitized and stored on a memory card on the battery powered headset, and signals were analyzed in custom software written in MATLAB (Math Works, Natick, MA, USA). Sleep profiling was accomplished using the Sleep Profiler (SP) (Advanced Brain Monitoring, Carlsbad, CA). Sleep stages were automatically detected from the device and edited in standard 30 sec intervals. The timing of SP and HP recordings were aligned and analyzed in register.

Results: In one particular clinical trial 18 volunteer subjects were consented to allow combined SP and HP recordings during a normal night of sleep. This included 9 female and 9 male subjects. Average age was 41.9 years (IQR 24.8-61.5). A total of 297 hours of combined HP and SP recordings were obtained. Headpulse recording revealed transient increases in forces beginning just prior to the onset of sleep determined by polysomnographic distribution and repeatable variation in occurrence over the sleep period. We termed this phenomenon as HeadPulse Sleep Bursts (HPSB). HPSBs were observed in 9/9 (100%) of subjects. The number of HPSBs in subjects with a full night of sleep ranged from 100-500. Overall, HPSB occurred at a mean frequency of 0.65 HPSB/min (SD 0.34), or (0.01 Hz) for the whole epoch of sleep but typically occurred in runs of every 20-50 seconds (0.02-0.05 Hz) interspersed with minutes of no bursts. In some subjects the timing of HPSB was remarkably periodic happening every 50 seconds in one subject and every two minutes in another. Using the awake period just prior to sleep as the reference, HPSB occurred 1.16, 1.42, 1.07, and 0.84 times more frequent in REM, N1, N2 and N3 sleep stages. The frequency of N2 stage HPSB frequency was marginally significant compared to awake (p=0.49, T-test). Normal subjects who had headpulse recording in the seated or supine position were at a rate of 0.1 bursts/minute, but it was difficult to separate gross body motion from what would be typical of a sleep burst.

Conclusions: This is the first report of HPSB phenomenon in humans or mammals. HPSB occurred in 100% of recorded subjects and is mostly independent of sleep stage. HPSB began during wakefulness just prior to sleep onset in all subjects and are not present in awake subjects not preparing for sleep. The relative low frequency of these bursts in the 20-50 second range (0.02-0.05 HZ) has no parallel in human sleep profiles but does match that seen with transient reversal of CSF flow in the cerebral aqueduct in sleeping humans measure on MRI, and the frequency of locus coeruleus discharges in sleep. The presence only during sleep indicates this is linked to mammalian glymphatic drainage and therefore could be a novel biomarker for this putative cleansing mechanism during sleep.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A method for determining or detecting a sleep brain signal in a human subject, comprising:
affixing an accelerometer against the cranium of a subject, such that the accelerometer remains on the subject through a cycle of sleep,
connecting the accelerometer to a computer to receive acceleration data from cranial accelerations correlated with heartbeats of the subject, and recording the acceleration data with the computer,
analyzing the recorded acceleration data to detect a particular signal distinct from surrounding signals and occurring at essentially regular intervals less than about one minute, and
if the particular signal is detected, comparing the particular signal data with library data of the particular signal from other human subjects to determine any deviation from a normal pattern, to detect an actual or potential brain anomaly.

2. The method of claim 1, wherein the particular signal comprises a burst of spikes higher in amplitude than surrounding time-domain acceleration data.

3. The method of claim 1, wherein the comparing step includes comparing frequency, number and power of the detected particular signal with the library data.

4. The method of claim 3, wherein the comparing step further includes measuring variances in width or amplitude during a subject's particular signals.

5. The method of claim 1, wherein the comparing step includes comparison of the human subject's particular data with signal data from one or more persons known to have at least early-stage dementia.

6. The method of claim 1, wherein the comparing step includes comparison of the human subject's particular data with signal data from one or more persons with PTSD.

7. The method of claim 1, wherein the comparing step includes comparison of the human subject's particular data with signal data from one or more persons known to have long-term concussion.

8. The method of claim 1, wherein the comparing step includes comparison of the human subject's particular data with signal data from one or more persons known to have mild cognitive impairment.

9. The method of claim 1, wherein the comparing step includes comparison of the human subject's particular data with signal data from one or more persons known to have particular neurodegenerative disorders.

10. A method for determining or detecting a sleep brain signal in a mammal subject, comprising:
affixing an accelerometer against the cranium of a subject, such that the accelerometer remains on the subject through a cycle of sleep,
connecting the accelerometer to a computer to receive acceleration data from cranial accelerations correlated with heartbeats of the subject, and recording the acceleration data with the computer,
analyzing the recorded acceleration data to detect a particular signal distinct from surrounding signals and occurring at essentially regular intervals less than about one minute, and
if the particular signal is detected, comparing the particular signal data with library data of the particular signal from other mammal subjects to determine any deviation from a normal pattern.
